(19) United States
(12) Patent Application Publication
(10) Pub. No.: US 2003/0147776 A1
(43) Pub. Date: Aug. 7, 2003

(54) Filtration of red blood cells

(76) Inventors: David F. Stroncek, Rockville, MD (US); Susan F. Leitman, Potomac, MD (US); Herbert M. Cullis, Gaithersburg, MD (US)

Correspondence Address: MERCHANT & GOULD PC, P.O. BOX 2903, MINNEAPOLIS, MN, 55402-0903, US

(21) Appl. No.: 10/289,686

(22) Filed: Nov. 6, 2002

Related U.S. Application Data
(60) Provisional application No. 60/338,806, filed on Nov. 6, 2001.

(51) Int. Cl. A61M 1/14; A61M 37/00; A61B 19/00; C02F 1/44

(52) U.S. Cl. 422/44; 604/6.02; 604/6.09; 604/6.03; 210/645; 604/403

(57) ABSTRACT

A method of reducing leukocytes in whole blood by collecting whole blood from a donor, increasing the oxygen level of the whole blood, wherein the whole blood includes RBC component and a remainder component, and filtering the RBC component to reduce the amount of leukocytes.

This application claims priority to U.S. Provisional Application Serial No. 60/338,806, filed Nov. 6, 2001 entitled Filtration of Red Blood Cells, the disclosure of which is incorporated herein by reference.

This invention is supported by the Department of Health and Human Services. The Government of the United States of America may have certain rights in the invention disclosed and claimed herein below.

### FIELD OF THE INVENTION

The invention generally relates to methods of reducing leukocytes in whole blood. More specifically, the invention relates to methods of producing leukocyte reduced red blood cells from whole blood by increasing the dissolved oxygen content of the whole blood and oxygen bound to hemoglobin.

### BACKGROUND OF THE INVENTION

Since 1998 the FDA's Blood Products Advisory Committee has recommended leukocyte reduction of all blood components. The reduction of the leukocyte content in cellular blood products may lead to a number of benefits, some of which include preventing alloimmunization, febrile reactions, cytomegalovirus infections, and transfusion associated immune suppression.

Generally, leukocytes are removed from RBC components during the processing of the blood through the use of filters specially designed for this purpose. These filters are highly effective, but approximately 1% of filtered RBC components still do not meet the criteria for leukocyte reduced red blood cell (“RBC”) component. In these instances, the quantity of leukocytes remaining in the RBC component, or the loss of RBC component is still too high.

Several preliminary studies have alleged that RBC components that do not meet the criteria for leukocyte reduction are more likely to be from people with sickle cell trait. One such study has found that approximately one half the RBC components collected from people with sickle cell trait occlude leukocyte reduction filters, one quarter pass completely through the filter (but the quantity of leukocytes remaining still exceed the criteria for leukocyte reduction), and only one quarter are successfully leukocyte reduced. Gorlin J B, et al., Transfusion 2000: 40 (supplement) 55S.

People with sickle cell disease are homozygous for hemoglobin S and experience chronic anemia, acute chest syndrome, stroke, pain crises, splenic dysfunction, and renal dysfunction due to the polymerization of hemoglobin S and RBC membrane changes. In contrast, people with sickle cell trait are heterozygous for hemoglobin S and experience no sickle cell symptoms.

Hemoglobin S in RBCs from people with sickle cell trait can polymerize at low oxygen tension, low pH, and high hemoglobin S concentration. Under physiological conditions the concentration of hemoglobin S in RBCs from people with sickle cell trait is not high enough to polymerize. However, whole blood collected by phlebotomy generally flows into bags with citrate anticoagulant. This renders the whole blood hyperosmotic and decreases the pH. These extreme conditions may damage the first portion of the blood collected and may result in a so called “citrate collection” lesion which can cause the polymerization of hemoglobin S. In addition, since blood is collected from veins, it has a low level of oxygen that can cause the polymerization of hemoglobin S. The combination of low oxygen levels and the citrate collection lesions can often result in an occlusion in the leukocyte reduction filter. When this occurs either the filter is less effective and the blood is unusable or the blood may clog the filter and may have to be disposed of.

Because of the need for leukocyte reduced RBC, and the large number of blood donors that may have sickle cell trait, there remains a need for an improved method of performing leukoreduction of RBCs on normal donor blood as well as sickle cell trait carrier blood that does not cause clogging and similar problems with the leukofiltration of the blood.

### SUMMARY OF THE INVENTION

In accordance with one aspect of the invention, there is provided a method of reducing leukocytes in whole blood by collecting whole blood from a donor, increasing the oxygen level of the whole blood, wherein the whole blood includes RBC component and a remainder component, and filtering the RBC component to reduce the amount of leukocytes.

In accordance with another aspect of the invention, there is provided a method of reducing leukocytes in whole blood that includes collecting whole blood from a donor, wherein the whole blood includes RBC component and a remainder component, separating the whole blood into the RBC component and the remainder component, increasing the oxygen level of the RBC component, and filtering the RBC component to reduce the amount of leukocytes.

The invention offers methods of collecting and filtering blood which can be utilized to effectively filter even sickle cell trait blood without clogging the filter by increasing the oxygen content of the blood. The invention offers a number of ways through which the oxygen content of the blood can be increased, including but not limited to, collecting the blood in oxygen permeable bags, shaking or agitating blood that has been collected in oxygen permeable bags, collecting the blood in an oxygen permeable bag with a higher than normal surface to volume ratio, increasing the time of storage before filtration, collecting or processing the whole blood with a system comprising oxygen permeable tubing, adding oxygen or air to the drawn whole blood, having the donor inhale oxygen from an oxygen mask, having the donor hyperventilate, or combinations thereof.

### BRIEF DESCRIPTION OF THE DRAWINGS

FIG. 1 illustrates a blood collection system in accordance with an embodiment of the invention.

FIGS. 2A, 2B, 2C, 2D, 2E, and 2F illustrate configurations of gas permeable tubing in accordance with the invention.

FIG. 3 illustrates another blood collection system in accordance with the invention.

FIG. 4 illustrates an aphaeresis system in accordance with the invention.

FIGS. 5A and 5B illustrate the oxygenation of blood stored in a PVC bag, a PL732 bag, and a Teflon bag.

FIGS. 6A and 6B illustrate the oxygenation of half units of blood to which 0 mL, 30 mL, or 60 mL of air has been added.

FIG. 7 illustrates post-filtration RBC recoveries for half units of sickle cell trait blood incubated for 2 hours with 60 mL and 0 mL of air.

### DETAILED DESCRIPTION OF THE PREFERRED EMBODIMENT

The invention comprises drawing whole blood from a donor, separating red blood cells from the whole blood, and reducing leukocyte concentration. The oxygen level may be increased in the whole blood prior to separation or in the red blood cell component after separation.

Methods of the invention provide for leukoreduction of whole blood comprising a step of collecting whole blood from a donor through use of, for example, phlebotomy techniques.

Methods and procedures for drawing blood are well known to those of skill in the art having read this specification. There are also a number of texts that offer details regarding such procedures. An example of such a text is The American Association of Blood Banks (AABB) Technical Manual, 13^{th} Ed., 1999, Bethesda, Md., which is incorporated herein by reference.

Another method of collecting whole blood from a donor is through aphaeresis. Aphaeresis is a procedure that separates at least one blood component from whole blood and returns the remainder blood to the donor.

Drawn whole blood can either be arterial blood or venous blood, depending on where (an artery or vein, respectively) the whole blood is removed from the donor. Generally methods of the invention draw whole blood from veins, and therefore, the drawn whole blood is venous blood.

Generally, venous blood whether drawn by phlebotomy or aphaeresis has oxygen levels of about 30 to 40 mm Hg. Oxygen levels of a certain amount of mm Hg refer to the partial pressure of O₂ in the blood. Generally, venous blood whether drawn by phlebotomy or aphaeresis has an oxygen saturation level of about 40%. An oxygen saturation level refers to the amount of hemoglobin that is carrying oxygen.

Methods of the invention comprise a step that increases the oxygen level and/or the oxygen saturation level of drawn whole blood. Examples of steps that increase the oxygen level of drawn whole blood include, but are not limited to, collecting the blood in oxygen permeable bags, shaking or agitating blood that has been collected in oxygen permeable bags, collecting the blood in an oxygen permeable bag with a higher than normal surface to volume ratio, increasing the time of storage before filtration, collecting or processing the whole blood with a system comprising oxygen permeable tubing, adding oxygen or air to the drawn whole blood, having the donor inhale oxygen from an oxygen mask, having the donor hyperventilate, or combinations thereof.

In one embodiment of the invention venous blood with oxygen levels of at least about 50 mm Hg, at least about 70 mm Hg, or an oxygen saturation level of at least about 80% are utilized. More preferably the venous blood has oxygen levels of at least about 90 mm Hg, or an oxygen saturation level of at least about 90%. Most preferably, the venous blood has oxygen levels of at least about 100 mm Hg, or an oxygen saturation level of about 100%.

Methods of the invention also comprise a step of filtering the collected blood with a leukocyte reduction filter. Leukocyte reduction filters generally function through one of two mechanisms, screen filtration, which depends on the size of the particles and depth filtration, which depends on one or more of three different mechanisms. In indirect adhesion, activated platelets are spread over and adhered to the filter. This causes attachment of granulocytes to the platelets. Direct adhesion of granulocytes and monocytes/macrophages surround the fibers of the filter and they are retained thereby. Mechanical sieving is a process that catches leukocytes, mononuclear cells and viable granulocytes in the fibers of the filter.

Some embodiments of methods of the invention also comprise a step of separating the whole blood into an RBC component and a remainder component. An RBC component as used herein generally comprises red blood cells. An RBC component also contains any white blood cells or leukocytes present in the sample. Leukocytes are generally thought to be undesirable. A remainder component generally contains constituents of blood other than red blood cells. A remainder component generally comprises plasma and may or may not comprise platelets.

Separating whole blood can be accomplished through a number of different methods. One method is to centrifuge the contents of the bag so that the RBC components are separated from the remainder component. Generally, this separation can be accomplished by centrifuging the bag containing the whole blood at speeds of from about 2000 to 5000 rpm. If it is desired to separate platelets from the RBC component, the whole blood is generally centrifuged at about 2000 rpm. If platelets are to remain in the RBC component, the whole blood is centrifuged at about 4000 rpm. Another method of separating the RBC component from the remainder component is through use of an aphaeresis system as mentioned above. An aphaeresis unit also separates components of whole blood with centrifugation principles.

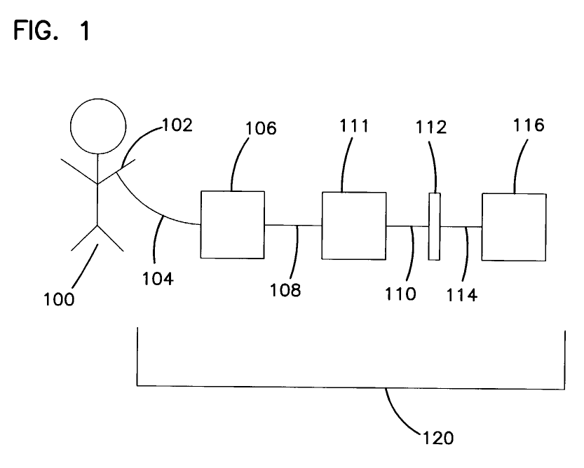

In one embodiment of the invention, the whole blood is drawn from a donor 100 and processed with a blood collection system 120. One example of a blood collection system 120 in accordance with the invention is depicted in FIG. 1. A commercially available example of a blood collection system in accordance with the invention is a RCM1 Leukotrap RC System from Medsep Corporation, Pall Medical, Corina, Calif. A blood collection system in accordance with the invention comprises blood drawing element 102, blood transfer line 104, first blood collection bag 106, first transfer line 108, preservative bag 111, filter input line 110, leukocyte reduction filter 112, filter output line 114, and RBC storage bag 116. Blood collection system 120 is generally used on a donor 100, which can be a mammal, preferably a human.

Blood drawing element 102 comprises an element that allows blood to be withdrawn from donor 100. Generally speaking blood drawing element 102 comprises a needle and a canula or an in-dwelling withdrawal tube. Preferably blood drawing element 102 comprises a 16 gauge stainless steel needle attached to plastic, preferably polyvinyl chloride tubing. Drawing blood from a donor 100 in this fashion is generally referred to as phlebotomy. Blood transfer line 104 comprises an element that transfers blood from blood collection element 102 to first blood collection bag 106. Generally speaking blood transfer line 104 comprises plastic tubing such as polyvinyl chloride. In one embodiment of the invention, the oxygen level of the drawn whole blood can be increased by using gas permeable tubing as described below.

Gas permeable tubing for use as blood transfer line 104 comprises plastic, for example, PVC, polyethylene, fluorocarbon, a multi-layer coextruded film, or combinations thereof. Preferably, blood transfer line 104 comprises a fluorocarbon. More preferably, blood transfer line 104 comprises fluoroethylenepropylene.

In embodiments of the invention where blood transfer line 104 is made of gas permeable tubing, the gas permeable tubing can be manufactured in any fashion that is similar to the manufacture of other types of gas tubing. After one of skill in the art has chosen the specific material for the gas permeable tubing, for example fluoroethylene-propylene, one of skill in the art would generally know how to construct such tubing. The diameter and lengths of tubing necessary would be dictated at least in part by the other components of the system it was to be used in.

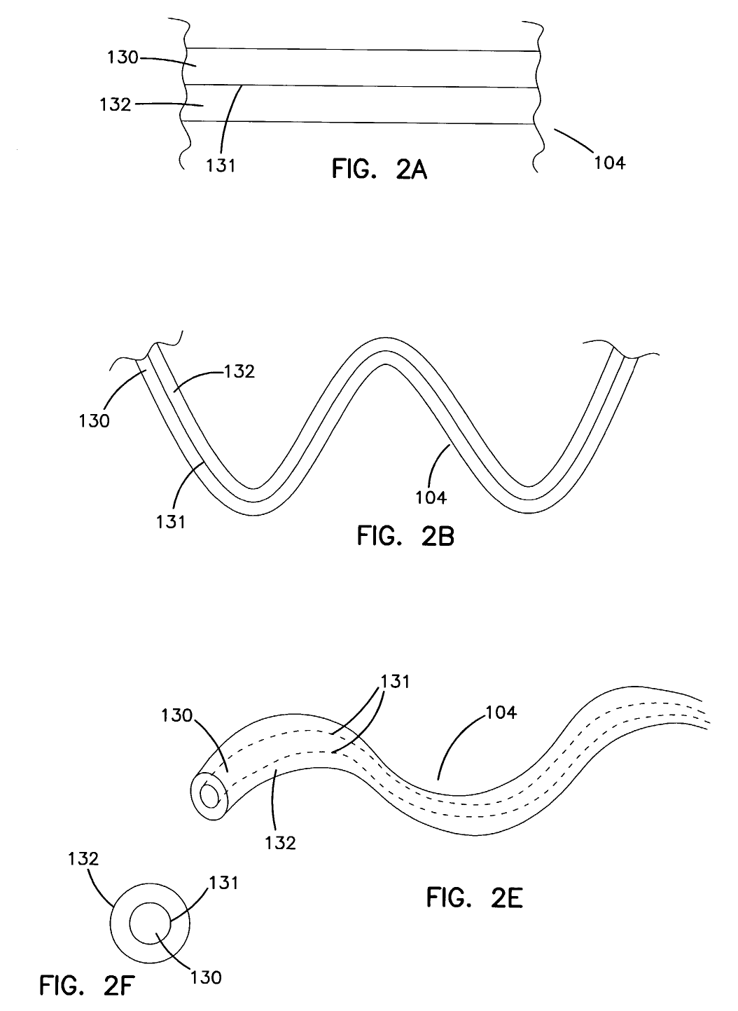

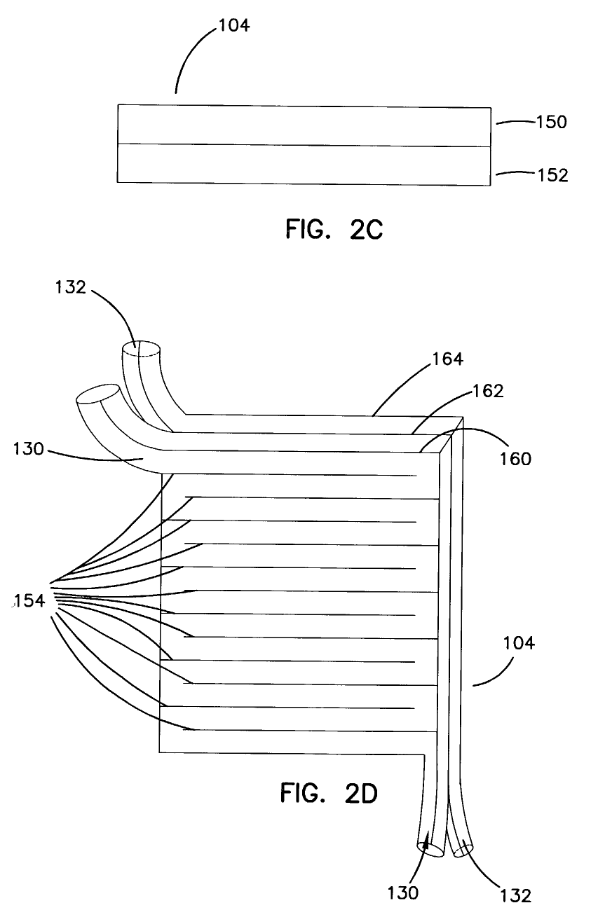

In a preferred embodiment, blood transfer line 104 further comprises at least two discrete portions of gas permeable tubing. An illustration of a blood transfer line 104 comprising two discrete portions of gas permeable tubing is depicted in FIG. 2A. One of the portions of gas permeable tubing would be blood line 130. Blood line 130 is configured for the whole blood to flow through. While the other portion of blood transfer line 104 would be gas line 132. Gas line 132 is configured for gas to flow through. Gas line 132 can be configured to have ambient air or oxygen flow through. Preferably, oxygen is flowing through gas line 132. Preferably, blood line 130 and gas line 132 would have a maximum area of contact at contact area 131. Preferably, contact area 131 would allow a maximum amount of the oxygen contained in gas line 132 to mix with the blood in blood line 130.

In an even more preferred embodiment, illustrated in FIG. 2B, blood transfer line 104 has a tortuous or serpentine pathway. Such a pathway preferably creates a maximum contact area 131 for gas to flow from gas line 132 to blood line 130. A tortuous pathway may also maximize the turbulence within the blood line 130, which maximizes the transfer of oxygen into the whole blood contained in blood line 130.

In yet another preferred embodiment, blood transfer line 104 is constructed of fluoroethylenepropylene, comprises at least two discrete portions one blood line 130 and gas line 132, having contact area 131 maximized and both portions having a common, serpentine pathway.

Another embodiment of this is seen in FIG. 2C. The blood transfer line 104 of FIG. 2c comprises at least two sheets of material a first sheet 150 a second sheet 152 which are fused together to create a serpentine path. At least one of the first sheet 150 and the second sheet 152 comprise a gas permeable material, fluoroethylepropylene for example. The fusion of first sheet 50 with second sheet 152 creates a pathway between the first sheet 150 and the second sheet 152. This pathway is used to flow the blood through. The blood transfer line 104 is then exposed to air, or preferably an oxygen rich environment, which allows oxygen to cross the at least one gas permeable barrier and increase the oxygen content of the blood. In a preferred embodiment, both the first sheet 150 and the second sheet 152 are made of gas permeable material, such as fluoroethylenepropylene.

In yet another embodiment, depicted in FIG. 2D, blood transfer line 104 is formed by fusing a first sheet 160, a second sheet 162, and a third sheet 164 together through the use of fusion lines 154 to form blood line 130 and gas line 132 as shown in FIG. 2e. This creates a desired serpentine pathway of blood line 130 and gas line 132 while maintaining maximum surface area contact across the second sheet 162. In one embodiment, at least second sheet 164, which forms the barrier between the blood line 130 and the gas line 132 is made of a gas permeable material, such as fluoroethylene-propylene. In another embodiment, the first sheet 160, the second sheet 162, and the third sheet 164 are all gas permeable materials, such as fluoroethylenepropylene. As a gas, air or preferably oxygen, is flowed through gas line 132, and blood is flowed through blood line 130, the oxygen from gas line 132 will cross the gas permeable barrier created by second sheet 162 and serve to increase the oxygen level of the blood. In embodiments having a serpentine pathway, the pathway serves to maximize the surface area at which blood line 130 contacts gas line 132, thereby increasing the transfer of oxygen across the gas permeable barrier of second sheet 162.

Yet another embodiment of blood transfer line 104 is depicted in FIGS. 2e and 2f. In this embodiment, blood line 130 is encased within gas line 132. It should also be understood that the alternative, could also be utilized, i.e. gas line 132 could be encased by blood line 130.

Referring again to FIG. 1, blood collection system 120 in accordance with the invention also comprises blood collection bag 106. Blood collection bag 106 functions to initially house whole blood drawn from donor 100. In one embodiment of the invention, blood collection bag 106 can also function to add anticoagulant to the drawn blood. For example, blood collection bag 106 can contain citrate as an anticoagulant. Generally, blood collection bag 106 comprises plastic. For example, blood collection bag 106 comprises polyethylene, polystyrene, or a fluorocarbon, such as fluoroethylepropylene, or mixtures of olefins, ethyls or ethylvinylacetates (EVAs), for example. Blood collection bag 106 can but need not be made of layers of plastics or laminates of plastic. Generally utilized blood collection bags which can be utilized for blood collection bags 106 in a blood collection system 120 of the invention include, but are not limited to those obtainable from Baxter Healthcare Corp., Deerfield Ill.; Pall Medical, Covina, Calif.; and Terumo Corp., Tokyo, Japan.

In one embodiment of the invention, the oxygen level of the blood can be increased by adding air or oxygen to the bag. The addition of air or oxygen can be accomplished prior to or after the blood is collected in the bag. In one embodiment of the invention, sterile air is injected into the bag after the blood is collected. Amounts of added air or oxygen can range from about 30 mL to about 150 mL per one unit of blood, preferably about 60 mL to about 140 mL per one unit of blood, more preferably about 120 mL per one unit of blood. To further increase the oxygen level of the blood, the bag containing the blood and air can be agitated or subject to storage times that are longer than the average 1 to 3 days.

In one embodiment of the invention, blood collection bag 106 is permeable to oxygen. Permeable bags that can be used in accordance with the invention have a number of characteristics. The material that the bag is constructed from must be biocompatible. Biocompatible means that the material is compatible with living tissue or a living system by not being toxic or injurious and not causing immunological rejection. The material that the bag is constructed of must also not have unacceptable levels of leaching, binding, adsorption or adherence. Unacceptable levels of leaching occur if biological materials enclosed within the bag ultimately contain a concentration level of molecules from the bag that renders the biological material unusable for its intended purpose. Unacceptable levels of binding, adsorption, or adherence occur if levels of critical components are decreased.

An example of a type of oxygen permeable bag that could be utilized as blood collection bag 106 include bags made from fluorocarbons. Fluorocarbons can be made into very thin sheets that have very high permeability. One example of a particular type of fluorocarbon formulation that can be used in methods of the invention is fluoroethylenepropylene (FEP). This type of fluorocarbon has very high permeability to oxygen. Another example of bags made from fluorocarbons include bags made from Teflon®.

Another example of a type of oxygen permeable bag that could be utilized as blood collection bag 106 in methods of the invention include those described in U.S. Pat. No. 6,297,046 B1 issued to Smith et al., which is hereby incorporated by reference in its entirety. These oxygen permeable bags are made from a multi-layer, co-extruded film. The film has an ultra-thin first layer of polystyrene with a thickness of from about 0.0001 inches to about 0.0010 inches. The second layer of the film is adhered to the first layer and is made of a polyolefin. The polyolefin acts as a flexible substrate for the polystyrene to provide a flexible, gas permeable film. The film can also have other layers that provide various characteristics to the bags such as strength or scratch resistance. In one preferred embodiment of the film of Smith et al. has an oxygen permeability of about 9-15 Barrers and a nitrogen permeability of about 10-100 Barrers.

Yet another example of an oxygen permeable bag that could be used as blood collection bag 106 in methods of the invention include polyvinyl chloride (PVC) bags. Generally, PVC bags have thicknesses of about 0.005 to about 0.15 inches. Preferably, these PVC bags are about 0.008 inches in thickness. Because the rate of gas transport across plastic is inversely proportional (in a linear fashion) to the thickness of the plastic, thinner walled bags such as these generally have higher gas transport rates and would therefore be more permeable to oxygen.

Even yet another example of an oxygen permeable bag for use as blood collection bag 106 in methods of the invention include polyethylene bags. Polyethylene bags have been shown to have an oxygen transport rate that is approximately twice as rapid as polyvinyl chloride bags. Therefore, polyethylene bags for use in the invention could be thicker than the PVC bags, discussed above. The extra thickness may be preferable, because it would tend to increase the strength of the bag.

Another example of a gas permeable bag is one in which one gas permeable bag is enclosed in another gas permeable, or non-gas permeable, bag. In this configuration, the outer non-gas permeable bag, for example, could contain oxygen gas that could transfer through the inner gas permeable bag. This would allow the oxygen to mix with the blood contained therein to increase the dissolved oxygen level. An example of a bag such as this is found in U.S. Pat. No. 4,455,299 issued to Grode, which is hereby incorporated by reference in its entirety.

One preferred example of an oxygen permeable bag for use in methods of the invention is a Lifecell Tissue Culture Flask with a 1000 mL capacity, available from Nexell Therapeutics, Inc., Irvine, Calif. 92618. These bags are made of a multi-layer co-extruded film of polystyrene and a polyolefin.

A number of different methods can be utilized in addition to utilizing a gas permeable bag to increase the oxygen level of the bag. In one embodiment, the bag that is utilized for collection of the blood can be larger than bags normally utilized for blood collection. For example, PVC bags normally utilized for blood collection generally have a capacity of about 0.6 L. In one embodiment of the invention, a PVC bag with a capacity greater than about 1 L is utilized, preferably greater than about 1.5 L, and more preferably from about 2 to about 3 L.

In one embodiment of the invention, the oxygen level of the blood can be increased by adding air or oxygen to the gas permeable bag. The addition of air or oxygen can be accomplished prior to or after the blood is collected in the bag. In one embodiment of the invention, sterile air is injected into the bag after the blood is collected. Amounts of added air or oxygen can range from about 30 mL to about 150 mL per one unit of blood, preferably about 60 mL to about 140 mL per one unit of blood, more preferably about 120 mL per one unit of blood. To further increase the oxygen level of the blood, the bag containing the blood and air can be agitated or subject to storage times that are longer than the average 1 to 3 days.

The time that the collected blood is stored before filtration can also be increased to increase the oxygen level of the blood. Standard protocols for blood collection and filtration generally provide for a 1 to 3 day delay before filtration of the blood. Methods of the invention increase this storage time to allow for an increased amount of oxygen that can cross the barrier of the gas permeable bag. Similarly, the gas permeable bag containing the collected blood can be agitated or shook to promote gas exchange and increase oxygen levels of the blood.

Furthermore, combinations of ways of increasing the oxygen level of the blood can also be utilized in methods of the invention.

Referring again to FIG. 1, blood collection system 120 in accordance with the invention also includes first transfer line 108. First transfer line 108 functions to transfers blood from blood collection bag 106 to preservative bag 111. Generally speaking first transfer line 108 comprises plastic tubing. In one embodiment, blood transfer line 108 comprises gas permeable tubing similar to blood transfer line 104.

One embodiment of a blood collection system 120, comprises preservative bag 111. Preservative bag 111 functions to add preservative to the blood and also contain the blood. Generally preservative bag 111 also functions to provide at least some cursory mixing of the preservative and the blood. Preservative bag 111 comprises a chemical or a solution that functions to preserve the blood. Examples of solutions that can be utilized to preserve the blood in preservative bag 111 include but are not limited to adenine-saline (AS-1) (contains NaCl, dextrose, adenine and other substances that support red cell survival), AS-3, or AS-5 (the 3 and 5 refer to different concentrations of various components in the solution). Preservative bag 111 further comprises plastic. For example, blood collection bag 106 comprises polyethylene, polystyrene, or fluoroethylenepropylene for example. In one embodiment of the invention, preservative bag 111 comprises gas permeable bags similar to blood collection bag 106.

Filter input line 110 functions to transfer blood from preservative bag 111 to leukocyte reduction filter 112. Generally speaking filter input line 110 comprises plastic tubing. In one embodiment, filter input line 110 comprises gas permeable tubing similar to blood transfer line 104.

The use of blood collection systems 120 in accordance with the invention are usually used along with a step to separate the red blood cell (RBC) component from the remainder component. Generally speaking this step is accomplished through centrifugation. The separation step can take place before or after the addition of preservative. Preferably, the preservative is added after the RBC component is separated from the remainder component. Generally speaking, blood collection bag 106 is configured to allow easy separation and removal of the remainder component from the RBC component after separation. In practice, this step is accomplished by centrifuging the blood collection bag and then removing the portion of it that contains the remainder component. Once the remainder component is separated from the RBC component, the blood collection system 120 can be utilized to accomplish leukocyte reduction of the RBC component.

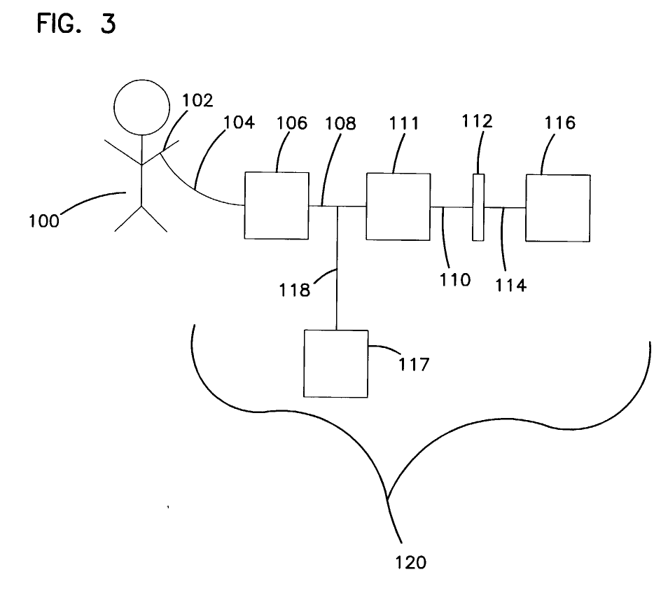

In another embodiment of the invention, a system, such as blood collection system 120 can be utilized to accomplish a method of the invention by adding oxygen into one of the lines or bags before the whole blood reaches the leukocyte reduction filter 112. An example of such an embodiment is depicted in FIG. 3. An exemplary system comprises the elements of blood collection system 120 and further comprises gas source 117 and gas transfer line 118. It should be understood that gas transfer line 118 can be attached to the system at any point before leukocyte reduction filter 112. Gas source 117 functions as a source of gas. Gas source 117 can either provide ambient air or oxygen. Preferably gas source 117 provides oxygen. Gas source 117 could be a tank of oxygen gas, a chemical reaction, air, or an attachment to a central source of oxygen. Preferably, gas source 117 is a tank of sterile oxygen gas or an attachment to a central source of oxygen. Gas transfer line 118 functions to transport gas from gas source 117 to the attached part of the system, in the case of FIG. 3, first transfer line 108.

In one embodiment of the invention, the amount of oxygen added to the blood would be effective to increase the oxygen level of the blood to at least about 50 mm Hg, preferably at least about 70 mm Hg, or at least about 80% saturated. More preferably, the amount of oxygen added would be effective to increase the oxygen level of the blood to at least about 90 mm of Hg or about 90% saturated. Most preferably, the amount of oxygen added to the blood would be effective to increase the oxygen level of the blood to about 100 mm Hg, or about 100% saturated.

The amount of oxygen added to the blood in a method of the invention in accordance with this embodiment could be regulated in a number of ways, including for example through regulation of the pressure or flow. For example, the flow of oxygen gas into gas transfer line 118 could range from about 1 to 10 L/min.

Methods of the invention generally function to increase the oxygen level of the whole blood withdrawn from donor 100 before the blood reaches the leukocyte reduction filter 112. In an embodiment of the invention, the oxygen level of the blood from donor 100 is increased at any one step in the process, for example, blood transfer line 104 could comprise a gas permeable line, or blood storage bag 106 could comprise a gas permeable line. In another embodiment of the invention, the oxygen level of the blood from donor 100 is increased at more than one step in the process, for example, blood transfer line 104, first transfer line 108, and first input line 110 could all comprise gas permeable lines, blood transfer line 104, and blood collection bag 106 could comprise a gas permeable line, and gas permeable bag respectively; or blood transfer line 104, blood collection bag 106, and first transfer line 108 could comprise a gas permeable line, a gas permeable bag, and a gas permeable line respectively. Methods of the invention encompass virtually any combinations of gas permeable lines, gas permeable bags, and other methods of increasing the oxygen level of the blood.

Blood collection system 120 comprises leukocyte reduction filter 112. Leukocyte reduction filter 112 functions to remove at least a portion of the leukocytes in the RBC component. Leukocyte reduction filter 112 generally comprise cotton, wool, cellulose, acetate, layers of non-woven webs of polyester fiber, microporous polyurethane, or combinations thereof. Generally, leukocyte reduction filters comprising polyester have layers with coarse pores at the inlet of the filter, layers with middle coarse pores in between, and layers with fine pores at the outlet of the filters.

To increase filtration mechanisms, physical and/or chemical modifications can be done.

Preferably, leukoreduction filters used in the invention comprise depth type filters.

In one embodiment of the invention, the process of leukoreduction is accomplished at lower temperatures, such as about 4° C.

In another embodiment, leukoreduced RBC components produced using a method of the invention have enhanced presentation characteristics. Leukoreduced RBC components with enhanced presentation characteristics refer to characteristics including but not limited to an ability to withstand longer storage times, an ability to withstand lower temperatures, or an ability to degrade less at similar temperature or storage time.

Filter output line 114 functions to transfer blood from leukocyte reduction filter 112 to RBC storage bag 116. RBC storage bag 116 comprises plastic. For example, RBC storage bag 116 comprises polyethylene, polystyrene, or fluorocarbons for example.

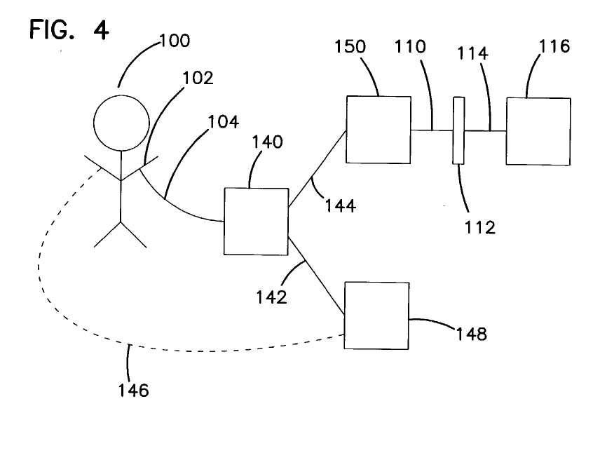

In another embodiment of the invention, blood is withdrawn from the donor 100 by aphaeresis. One embodiment of an aphaeresis system in accordance with the invention is depicted in FIG. 4. An aphaeresis system in accordance with the invention comprises blood withdrawal element 102, blood transfer line 104, aphaeresis unit 140, plasma line 142, plasma storage 148, optional return line 146, RBC component line 144, RBC component storage bag 150, filter input line 110, leukocyte reduction filter 112, filter output line 114, and RBC storage bag 116. Elements with the same function are named and numbered similarly and will not be discussed again.

Aphaeresis unit 140 functions to separate the RBC component from the plasma/platelet component. Aphaeresis unit 140 can comprise any system generally known to those of skill in the art, and includes for example, COBE spectra aphaeresis system (Gambro® BCT™ Inc., Blood Component Technology, Lakewood, Colo.), Trima Collection System (Gambro® BCT™ Inc., Blood Component Technology, Lakewood, Colo.), CS 3000 Plus (Fenwal, a division of Baxter Healthcare Corp., Deerfield, Ill.), Amicus cell (Fenwal, a division of Baxter Healthcare Corp., Deerfield, Ill.), MCS®+Apheresis System (Haemonetics®, Braintree, Mass.), and ALYX System (Fenwal, a division of Baxter Healthcare Corp., Deerfield, Ill.). In a preferred embodiment, aphaeresis unit 140 comprises a blood cell separator (MCS+®, Haemonetics Inc, Braintree, Mass.). Apheresis unit 140 may alternatively be comprised of gas permeable bags, or gas permeable tubing as discussed above.

Apheresis unit 140 is connected to remainder component line 142 and RBC component line 144. Remainder component line 142 functions to transport the remainder component to an optional remainder unit 148, and/or eventually to return line 146. The optional remainder unit 148 can be used to temporarily store the remainder component, store the remainder component for an extended period of time, treat the remainder component, or direct it back through return line 146 into donor. RBC component line 144 functions to transport the RBC component from aphaeresis unit 140 to optional RBC holding bag 150. In one embodiment of the invention, RBC component line 144 comprises gas permeable tubing as discussed above.

Optional RBC holding bag 150 functions to contain the RBC component if the aphaeresis system is configured so that this step is necessary or desirable. Alternatively, RBC holding bag 150 can be similar to preservative bag 111. In this embodiment RBC holding bag 150 functions to add preservative to the blood and also contain the blood. Generally RBC holding bag 150 also functions to provide at least some cursory mixing of the preservative and the blood. In this embodiment, RBC holding bag 150 comprises a chemical or a solution that functions to preserve the blood. Examples of solutions that can be utilized to preserve the blood in RBC holding bag 150 include but are not limited to adenine-saline (AS-1, AS-3, or AS-5). RBC holding bag 150 further comprises plastic. For example, blood collection bag 106 comprises polyethylene, polystyrene, or fluorocarbons for example. In one embodiment of the invention, RBC holding bag 150 comprises gas permeable bags similar to blood collection bag 106.

The remaining elements of the aphaeresis system illustrated in FIG. 3 are similar to those shown for the blood collection system of FIG. 1, and will not be discussed further.

### WORKING EXAMPLES

The following examples provide a non-limiting illustration of various embodiments of the invention.

These studies were approved by a NIH Institutional Review Board and informed consent was obtained before the blood was collected. Donors met all AABB criteria for donating whole blood. Sickle cell trait was confirmed by ion exchange high performance liquid chromatography (HPLC) analysis of donor RBCs (Variant HPLC system, β-thalassemia short program, BioRad Diagnostics Group, Hercules, Calif.). All donors were asked to disclose their current smoking status.

Blood counts were measured with a automated cell counter (Cell-Dyn 4000, Abbott Diagnostics, Santa Clara, Calif.). Blood gases, pH, and sodium, potassium, chloride, bicarbonate, and glucose levels were measured with a blood gas analyzer (Radiometer ABL 700 Series, Radiometer Analytical SA, Lyon, France or i-STAT Portable Clinical Analyzer, i-STAT Corporation, East Windsor, N.J.). Osmolarities were measured with a PSI-Multi-Osmette Model 2430 instrument (Precision Systems, Inc., Natick, Mass.).

Values represent the mean one standard deviation. Groups were compared using Student's t-tests. In some cases paired t-tests were used.

### EXAMPLE #1

### Filtration of RBCs Collected in CP2D

The filterability of blood from 6 sickle trait donors collected in CP2D (a solution of citrate-phosphate-dextrose-dextrose) was compared to the filterability of blood from the same donors collected in heparin.

RBCs were collected into a modified collection bag set that included CP2D anticoagulant, AS-3 (adenine-saline) additive solution, and a RBC leukocyte reduction filter (RCM1, Leukotrap RC System, Medsep Corporation, Pall Medical, Covina, Calif.). The set was changed by removing half of the CP2D (31 mL) from the collection bag, removing half of the AS-3 (50 mL), adding an additional collection bag that contained sodium heparin (2.5 mL, 1000 Units/mL, Elkins-Sinn Inc, Cherry Hill, N.J.), adding a bag containing AS-3 additive solution (50 mL), and adding a second leukocyte reduction filter (RCM1, Pall Medical). From each donor a volume of 250 mL of whole blood was collected into the bag containing CP2D and 250 mL was collected into the bag with heparin. The bags were rocked during collection (Sebra, Tucson, Ariz.).

Packed RBCs were prepared from components collected in CP2D and heparin. The packed RBCs were prepared by centrifuging the blood collection bag at about 4500 RPM. The packed RBCs were filtered according to the manufacturers instructions except only one-half the AS-3 additive solution (50 mL) was added to the packed RBCs. Samples were taken before and after the addition of AS-3 and after filtration for measurement of complete blood counts, osmolarity, blood gases, and pH.

RBC components from 6 people with sickle cell trait were studied. All the donors were healthy and met the criteria for blood donors. Their mean age was 46 years and ranged from 32 years to 53 years, one was male and all were African American. The percent of hemoglobins ranged from 33.7% to 39.0%. One of the sickle cell trait donors smoked cigarettes.

CP2D RBC components collected from 5 of the 6 donors with sickle cell trait occluded the filter before all the RBCs passed through the filter (Table 1). Among the 5 donors whose RBCs occluded the filter, no RBCs from 2 donors passed into the collection bag. The sickle trait donor whose CP2D RBC component filtered completely was the only one that smoked cigarettes and filtration time was 72 minutes. The RBC recovery of this donor's RBC component was 71% and the residual leukocyte count was 0.11×10⁶.

TABLE 1
Filtration of RBC Components from Donors with Sickle
Trait Collected in CP2D

| | | | Initial | Volume | | RBC | Residual |
|---|---|---|---|---|---|---|---|
| | Hgb S | Filtration | Volume | Filtered* | Filtration | Recovery | WBC |
| Donor | (%) | Outcome | (mL) | (mL) | Time(min) | (%) | (× 10⁵) |
| 1 | 35.7 | Obstructed | 155 | 53 | >120 | 34 | NA |
| 2 | 39.0 | Obstructed | 120 | none | >120 | 0 | NA |
| 3 | 35.4 | Complete | 147 | 123 | 72 | 71 | 0.11 |
| 4 | 38.8 | Obstructed | 113 | 58 | >120 | 26 | NA |
| 5 | 34.3 | Obstructed | 156 | none | >120 | 0 | NA |
| 6 | 33.7 | Obstructed | 129 | 72 | >120 | 40 | NA |

NA = Not Applicable
*Volume of component passing through the filter

RBC components were collected from 6 healthy African Americans without hemoglobin S; 3 were male and their mean age was 39 years and ranged from 32 years to 49 years. All 6 control components collected in CP2D filtered completely (data not shown). The mean filtration time was 18±5 minutes and ranged from 11 to 26 minutes. The residual leukocyte counts in all 6 components was less than 0.15×10⁶. The RBC recovery of the CP2D components collected from the control donors was greater than the RBC recovery of units collected from people with sickle cell trait (26% ±27% versus 82%±4%, p<0.004).

To determine if CP2D contributed to problems with filtering sickle cell trait donor RBC components, one-half unit of blood was collected into heparin from the same 6 donors with sickle cell trait. All 6 sickle trait donor heparin RBC components collected by phlebotomy filtered completely (Table 2). The residual leukocyte count in the 6 components was less than 1×10⁶ cells. The RBC recoveries of the 6 heparin RBC components from sickle trait donors was greater than the RBC recoveries of the 6 CP2D components collected from the same donors (78%±10% compared 26%±27%, p<0.005).

TABLE 2
Filtration of Sickle Trait RBC Components
Collected in Heparin

| | | Initial | | RBC | |
|---|---|---|---|---|---|
| | Filtration | Volume | Filtration | Recovery | Residual |
| Donor | Outcome | (mL) | Time (min) | (%) | WBC (× 10⁶) |
| 1 | Complete | 131 | 20 | 96 | 0.06 |
| 2 | Complete | 123 | 54 | 75 | 0.04 |
| 3 | Complete | 152 | 28 | 68 | 0.5 |
| 4 | Complete | 116 | 25 | 69 | 0.13 |
| 5 | Complete | 137 | 16 | 80 | 0.31 |
| 6 | Complete | 112 | 12 | 83 | 0.11 |

NA = Not Applicable
* Volume of component passing through the filter

RBC components from the 6 donors without hemoglobin S (normal African Americans) were also collected in heparin and filtered. When the RBC recoveries and filtration times were compared between the 6 components collected in heparin from donors with sickle cell trait and from the 6 components collected from control donors, no differences were found in filtration time (26±15 minutes versus 11±5 minutes, p=0.07) and red cell recoveries (78%±10% versus 91%±10%, p=0.10).

The pH of sickle cell trait components collected in CP2D (6.91±0.11) was lower than the pH of sickle cell trait RBCs components collected in heparin (7.10±0.01, p<0.03) when measured after AS-3 solution was added, but not when measured in whole blood immediately after collection (7.22±0.14 in CP2D and 7.27±0.12 in heparin). Similarly, the osmolarity of sickle cell trait RBC components collected in CP2D (316±12 mOsm/kg) was greater than the osmolarity of sickle cell trait RBC components collected in heparin (285±2 mOsm/kg, p<0.03) when the components were tested after adding AS-3 but not when tested immediately after collection (300±20 mOsm/kg in CP2D and 291±4 mOsm/kg in heparin). There was no difference in oxygen tension, hemoglobin oxygen saturation, or RBC mean cellular hemoglobin concentration (MCHC) between the sickle cell trait components collected in CP2D and heparin either immediately after collection or after adding AS-3. The similarities of these parameters among the RBC components collected in CP2D and heparin suggests a citrate collection lesion was contributing to the filter failures.

Blood chemistry levels, pH, and osmolarities were measured in whole blood immediately after the collection were compared among components collected from donors with sickle cell trait and those collected from control donors without hemoglobin S (Table 3). When components were collected in CP2D, sodium, glucose, and osmolarity were lower and chloride, potassium, and pH were greater in components collected from donors with sickle cell trait than in control donors without hemoglobin S. There was no difference in sodium, chloride, glucose, osmolarity, and pH among RBC components from the two groups collected in heparin. The marked differences in these parameters among sickle trait and control components collected in CP2D, but not those collected in heparin, suggests that CP2D had a greater effect on RBCs from donors with sickle cell trait than with control donors.

When blood is collected into citrate anticoagulant, RBCs swell. As expected, when control donor RBC indices were compared between RBC components collected CP2D and those collected in heparin, mean cellular volume (MCV) was greater in CP2D components and MCHC was less in CP2D components (Table 3). In contrast, no change in RBC volume occurred when sickle cell trait RBCs were collected into CP2D. There was no difference in sickle cell trait donor RBC MCV or MCHC between RBC components collected in CP2D and those collected in heparin.

Table 3. Comparison Between Donors with Sickle Cell Trait and Donors Without Sickle Cell Hemoglobin of Chemistry Levels in Whole Blood Components Collected in CP2D and Heparin

| | | | Heparin | |
|---|---|---|---|---|
| | CP2D | | | No-Sickle |
| | Sickle Cell | No-Sickle Cell | Sickle Cell | Cell |
| | trait | Hgb | trait | Hgb |
| | (n = 6) | (n = 6) | (n = 6) | (n = 6) |
| Sodium | 142 ± 9 | 152 ± 4* | 138 ± 2 | 139 ± 2 |
| Potassium | 4.5 ± 0.9 | 3.2 ± 0.2* | 4.8 ± 0.7 | 4.1 ± 0.2 |
| Chloride | 97 ± 9 | 80 ± 3* | 105 ± 1 | 105 ± 1 |
| Glucose | 277 ± 197 | 603 ± 88* | 100 ± 21 | 100 ± 14 |
| Osmolarity | 300 ± 20 | 335 ± 7* | 291 ± 4 | 297 ± 5 |
| pH | 7.22 ± 0.14 | 6.97 ± 0.08* | 7.27 ± 0.12 | 7.29 ± 0.04 |
| MCV | 84.3 ± 3.6 | 91.2 ± 6.7♯ | 83.9 ± 3.1 | 87.4 ± 5.1 |
| MCH | 28.7 ± 1.2 | 28.8 ± 2.0 | 28.4 ± 1.4 | 28.9 ± 1.6 |
| MCHC | 33.4 ± 9 | 31.6 ± 1.5♯ | 33.8 ± 0.6 | 33.0 ± 0.9 |

Chemistries and RBC indicies were measured in Components at the time of collection.
*p < 0.02 for comparison of CP2D blood collected from Sickle Cell Trait donors and donors without Sickle Cell Trait
♯p < 0.02 for comparison of CP2D and heparin components collected from donors without hemoglobin S

### EXAMPLE #2

### Filtration of Carbon Monoxide-Treated RBC Components

Blood collected in CP2D from 3 other sickle trait donors was divided in two and one-half was treated with carbon monoxide to convert hemoglobin S to its liganded form to prevent hemoglobin S polymerization. All 3 carbon monoxide-treated components filtered within 7 minutes, but only 1 of 3 untreated components filtered completely.

One unit of blood was collected in CP2D, packed RBCs were prepared, and AS-3 was added. The RBC component was then divided in half. One half was filtered as described above (RCM1, Pall Medical) and the other half was treated with carbon monoxide before filtration. To treat the component with carbon monoxide one half was added to a tonometer (Fisherbrand septum-port gas sampling tube, 250 mL, Fisher Scientific, Pittsburgh, Pa.) and carbon monoxide (Aldrich, St. Louis, Mo.) was allowed to slowly flow through the tonometer at room temperature for 60 minutes as it was gently rocked in an exhaust hood. After 1 hour of incubation with carbon monoxide the RBCs were transferred from the tonometer to a bag and filtered with a RBC leukocyte reduction filter (RCM1, Pall Medical). The filter was primed with AS-3 solution, but rather then adding the AS-3 to the RBCs after it passed through the filter, as the AS-3 left the filter it was diverted to an empty bag.

To determine if hemoglobin S polymerization was responsible for the occlusion of leukocyte reduction filters, RBC components were treated with carbon monoxide to convert hemoglobin S to its liganded form that prevents polymerization. RBC components collected in CP2D from 4 donors with sickle cell trait were divided in half; one-half was treated with carbon monoxide before filtering and the other half was filtered without further treatment (Table 4). All 4 RBC components treated with carbon monoxide filtered completely, but only 1 of the 4 untreated components filtered completely. In addition, among the six CP2D RBCs components collected during the first part of these studies, 2 did not filter at all. One component was hemolyzed and was not tested further, but the other component was treated with carbon monoxide and filtered again. Following carbon monoxide treatment, this component filtered completely in 7 minutes, its RBC recovery was 85%, and the residual leukocyte counts was 0.04×10⁶ cells. The RBC recoveries of the 5 carbon monoxide-treated sickle trait donor components were significantly greater than those of the untreated components from the same donors (84%±4% vs. 32%±36%, P<0.04).

TABLE 4
Filtration of Sickle Trait RBC Components Collected in
CP2D and Treated with Carbon Monoxide

| Untreated Components | | | | | Carbon Monoxide Treated Components | | | |
|---|---|---|---|---|---|---|---|---|
| | | Filtration | RBC | Residual | | Filtration | RBC | Residual |
| | Filtration | Time | Recovery | WBC | Filtration | Time | Recovery | WBC |
| Donor | Outcome | (min) | (%) | (× 10⁶) | Outcome | (min) | (%) | (× 10⁶) |
| 7 | Complete | 26 | 79 | 0.715 | Complete | 4 | 84 | 0.01 |
| 8 | Obstructed | >120 | 0 | NA | Complete | 7 | 88 | 0.01 |
| 9 | Obstructed | >120 | 19 | NA | Complete | 9 | 76 | 0.26 |
| 10 | Obstructed | >120 | 62 | NA | Complete | 9 | 86 | 0.01 |

### EXAMPLE #3

### Filtration of Apheresis RBC Components

Apheresis RBC components contain less CP2D and 5 of 7 sickle cell trait aphaeresis components filtered completely; 4 of the 5 filtered rapidly, less than 15 minutes, and one filtered in 100 minutes. Hemoglobin oxygen saturation was greater in the 4 rapidly filtering components (68%±9%) than the 3 filtering slowly or incompletely (37%±5%, p=0.03).

RBCs were collected using a blood cell separator (MCS+®, Haemonetics Inc, Braintree, Mass.) according to the manufacturer's recommendations except that only one unit of RBCs was collected. The CP2D anticoagulant was added to whole blood ratio of 1 to 16. Immediately after the RBCs were collected the blood cell separator added the RBCs to a bag containing 100 mL of AS-3.

The aphaeresis RBC component was divided into two. One half was filtered within two hours of collection using a RBC leukocyte reduction filter (RCM1, Pall Medical). Prior to filtering the RBCs, the filter was primed with AS-3 solution, but rather then adding the AS-3 to the RBCs after the AS-3 passed through the filter as the AS-3 left the filter it was diverted to an empty bag and discarded. When aphaeresis components filtered poorly, the second half unit was incubated in oxygen permeable blood bag for 2 hours at room temperature (Lifecell Tissue Culture Flask, 1000 mL capacity, Nexell Therapuetics, Inc., Irvine, Calif., 92618) before being filtered (RCM1, Pall Medical).

Apheresis RBC components were collected from 7 healthy people with sickle cell trait. All 7 donors were African American, their median age was 39 years and ranged from 20 to 50 years, and 2 were male. Five of the seven sickle trait aphaeresis RBC components passed completely through the leukocyte reduction filter. Four of the five aphaeresis RBC components filtered in less than 15 minutes and 1 filtered in 100 minutes. The residual leukocyte count in all 5 components filtering completely was less than 0.9×10⁶ cells and residual RBCs recoveries in all 5 was greater than or equal to than 85% (Table 5). When the RBC recovery of components collected from sickle cell trait donors by aphaeresis was compared to components collected from sickle cell trait donors by phlebotomy into CP2D, RBC recover was greater in the aphaeresis RBC components (67%±35% versus 28%±27%, p<0.05).

TABLE 5
Filtration of RBC Components Collected by Apheresis from
Donors with Sickle Trait

| | | | Initial | | RBC | |
|---|---|---|---|---|---|---|
| | Hgb S | Filtration | Volume | Filtration | Recovery | Residual |
| Donor | (%) | Outcome | (mL) | Time (min) | (%) | WBC (× 10⁶) |
| 1 | 39.0 | Complete | 162 | 12.1 | 86 | 0.41 |
| 2 | 37.3 | Complete | 162 | 8.0 | 85 | 0.04 |
| 3 | 30.4 | Complete | 160 | 9.8 | 91 | 0 |
| 4 | 39.2 | Obstructed | 155 | >120 | 13 | 0.25 |
| 5 | 38.3 | Complete | 158 | 6.3 | 93 | 0.89 |
| 6 | 39.2 | Obstructed | 160 | >120 | 17 | 0.03 |
| 7 | 35.1 | Complete | 160 | 100 | 85 | 0.24 |

NA = Not Applicable
* Volume of component passing through the filter

As a control, aphaeresis RBC components were collected from 4 healthy donors without hemoglobin S; 3 were African Americans, one Caucasian, and all were male. Their mean age was 38 years and ranged from 28 to 46 years. For all 4 control aphaeresis RBCs components, filtration time was less than 10 minutes, RBC recovery was greater than 87%, and residual leukocyte counts were less than 0.3×10⁶ cells. The mean filtration time was 8±2 minutes, the RBC recovery was 91±3%, and residual leukocyte count was 0.2±0.2×10⁶ cells. RBC recoveries were similar for aphaeresis RBC components collected from donors with sickle cell trait and from donors without hemoglobin S (67%±35% versus 91%±32%, p=0.22).

Although several differences were found in the properties of CP2D components collected by phlebotomy among sickle cell trait and control donors, there were no differences among aphaeresis units collected from sickle cell trait and control donors in chemistries and RBC indices or oxygen saturations (sodium, potassium, chloride, glucose, osmolarity, pH, PO₂, oxygen saturation, osmolarity, MCV, MCH, and MCHC) (data not shown).

The two components that occluded the filter and the one that completed filtration in 100 minutes were considered “poorly” or “slowly” filtering components. The properties of the 4 aphaeresis components that filtered “rapidly”, less than 15 minutes, were compared with those of the 3 units that filtered poorly. There was no difference in hemoglobin S fraction, pH, or MCHC between the two groups, but the oxygen saturation was greater in the rapidly filtering group (68%±9% versus 37%±5%, p<0.03).

### EXAMPLE #4

### Effect of Incubating RBC Components in Gas Permeable Bags

The unfiltered one-half aphaeresis RBC unit from the 3 sickle cell trait donors whose aphaeresis RBC components filtered poorly were incubated for 2 hours in bags that were more gas permeable than typical whole blood collection bags in order to attempt to convert hemoglobin S to its liganded configuration. The incubation increased the hemoglobin oxygen saturation from 36.9%±4.7% to 60.3%±12.0% (p<0.04) (Table 6).

TABLE 6
Filtration of Apheresis RBC Components from Donors with Sickle Trait that
were incubated in Oxygen Permeable Bags for 2 hours at 22° C.

| | Pre-Incubation | Post-Incubation | Filtration | Filtration | RBC | Residual |
|---|---|---|---|---|---|---|
| Donor | O₂ sat. (%) | O₂ sat. (%) | Outcome | Time (min) | Recovery (%) | WBC (× 10⁵) |
| 4 | 42.3 | 74 | Complete | 7.9 | 87 | 0.80 |
| 6 | 33.4 | 56 | Obstructed | >120 | 42 | NA |
| 7 | 35.1 | 51 | Complete | 8.9 | 94 | 0.80 |

NA = Not Applicable

Two of the three components incubated in gas permeable bags filtered completely and the post-filtration RBC recovery increased from 38%±39% to 74%±28% but not enough donors were studied to demonstrated a significant change in RBC recovery (p=0.20).

Polymerization of hemoglobin S during the collection and processing of blood appears to be responsible for the ineffective performance of RBC leukocyte reduction filters with RBC components collected from donors with sickle cell trait. When hemoglobin S polymerizes, RBC intracellular viscosity increases, reducing deformability, and impairing filterability. The trapping of RBCs with polymerized hemoglobin S in leukocyte reduction filters leads to either the complete obstruction of flow or the channeling of flow that makes filtration ineffective. Treating the RBCs to increase hemoglobin oxygen or carbon dioxide levels converted hemoglobin to its liganded form which prevented hemoglobin S polymerization and allowed the successful filtration of sickle cell trait donor RBC components.

These findings are significant because the polymerization of hemoglobin S in RBCs from donors with sickle cell trait has not been thought to be of clinical relevance and not responsible for the occlusion of leukocyte reduction filters. In most clinical situations hemoglobin oxygen saturations are high enough to prevent hemoglobin S polymerization in people with sickle cell trait. As a result it has not been expected that hemoglobin S polymerization was responsible for filter failures in blood donors with sickle cell trait. This study shows that conditions in the blood collection bags can cause hemoglobin S polymerization.

In some clinical situations the polymerization of hemoglobin S in RBCs from people with sickle cell trait is important. In the renal medulla where oxygen tension and pH are low and osmolarity is high, hemoglobin S polymerizes leading to microvascular occlusion and the impaired ability to concentrate urine. The high osmolarity draws intracellular water from RBCs increasing the hemoglobin S concentration. The increase in hemoglobin S concentration and the low oxygen tension can result in hemoglobin S polymerization. In addition, during extreme physical stress, hemoglobin S polymerization can occur in people with sickle cell trait. During the collection of blood by phlebotomy, RBCs are exposed to conditions that result in the polymerization of hemoglobin S.

Although several factors affect the polymerization of hemoglobin S, an important cause of hemoglobin S polymerization in RBC components is likely the collection of blood into the citrate anticoagulant solution. While all 6 RBC components collected into heparin were effectively leukocyte reduced by filtration, only one of 6 units collected into citrate anticoagulant was effectively leukocyte reduced. When whole blood is collected by phlebotomy it is collected into 63 mL of CP2D or a similar citrate-based anticoagulant. CP2D has an osmolarity of 585 mOsm/kg and a pH of 5.7 and the low pH and high osmolarity of CP2D along with the low oxygen saturation of venous blood favor hemoglobin S polymerization. The effects of citrate anticoagulant are likely most marked on the first few milliliters of blood collected. We hypothesize that these conditions result in hemoglobin S polymerization in at least the first portion of blood collected if not the entire component.

Further evidence of a citrate collection lesion and of hemoglobin S polymerization in sickle trait blood collected in CP2D is the difference in chemistries and RBC indices in CP2D components collected by phlebotomy between donors with sickle cell trait and control donors. Sodium, potassium, chloride, glucose and osmolarity level and pH differed in CP2D blood collected from donors with sickle cell trait and those without hemoglobin S. We speculate that this is due to the polymerization of hemoglobin S. When hemoglobin S polymerizes the intracellular osmolarity falls drawing glucose intracellularly and reducing the extracellular glucose levels and osmolarity. The fact that no difference in osmolarity or glucose levels occurred in blood collected in heparin supports that CP2D collection lesion is responsible for hemoglobin S polymerization.

We found that aphaeresis RBCs components collected from sickle trait donors filtered more effectively than RBC components collected by phlebotomy into CP2D. The improved filterability of aphaeresis RBC components compared to phlebotomy components is likely due to avoidance of the citrate collection lesion. In contrast to phlebotomy CP2D components, no differences in the properties of aphaeresis components collected from sickle trait and control donors were noted indicating that polymerization was less problematic in aphaeresis units. The reduction or elimination of the citrate collection lesion in aphaeresis RBC components is likely due to the method of addition of CP2D. During aphaeresis CP2D is added to the blood at a carefully controlled rate proportional to the whole blood collection rate. In contrast with phlebotomy collections of blood flows into a bag containing enough CP2D to anticoagulate an entire unit.

Other factors in addition to a citrate collection lesion also contributed to hemoglobin S polymerization. Although filtration of aphaeresis RBC components was improved compared to phlebotomy components, some aphaeresis components did not filter effectively. The components that did not filter effectively had lower hemoglobin oxygen saturations levels that those that did filter completely. It is unclear as to why hemoglobin oxygen saturation levels vary among donors, but incubation of aphaeresis components in gas permeable bags prior to filtration increased hemoglobin oxygen saturation and improved the filterability of sickle trait RBCs. However, one component occluded a filter after incubation in gas permeable bags at room temperature. Since hemoglobin S is less likely to polymerize at lower temperatures, the combination of incubating aphaeresis RBC components in gas permeable bags and reducing its temperature to 4° C. may be more effective at preventing hemoglobin S polymerization and filter failure than simply incubating the RBC component in gas permeable bags. It is likely that incubating phlebotomy CP2D RBC components in gas permeable bags at 4° C. will also improve the filterability of these components, but the fact that one CP2D phlebotomy component hemolyzed, suggests that it will not be possible to successfully filter all sickle trait components collected by CP2D phlebotomy.

Blood from sickle trait donors collected in heparin filtered effectively, but heparin is not a suitable anticoagulant. An alternative to collecting blood in CP2D is the collection in an alternative citrate anticoagulant such as citrate-phosphate-dextrose (CPD) or citrate-phosphate-dextrose-adenosine (CPDA-1) solutions. The osmolarity of CPD and CPDA-1 is less than that of osmolarity of C2PD since they contain less dextrose than CP2D. However, CPD and CPDA-1 are also hyperosmotic and acidic and RBC filter performance problems have been reported with RBC components from sickle trait donors collected in CPDA-1.

These studies have several implications. Since filter failures were due to changes in the rheological properties of the sickle cell trait RBCs, it would be expected that without some intervention to prevent hemoglobin S polymerization, RBC components from donors with sickle cell trait would occlude, most, if not all leukocyte reduction filters. The incidence of failure may vary among filters depending on the construction of the filter and filter material, the type of citrate anticoagulant, the temperature of the blood at the time of filtration, and the interval of storage before blood was filtered. It would be expected that RBC components cooled to 4° C. would filter better than those filtered at room temperature. In addition, it is likely that RBC components stored for several days or weeks in gas permeable bags prior to filtration would have increased oxygen tensions and filter more effectively.

These results show that when blood from donors with sickle cell trait is collected under the appropriate conditions, filters can be used to remove leukocytes from RBC components. It is important to find existing collection systems or develop new systems that permit the leukocyte reduction of RBCs from donors with sickle cell trait. Sickle cell trait is most prevalent in African Americans and since this population is under represented among blood donors it important not to exclude people with sickle cell trait from donating any blood component.

### EXAMPLE #5

### Comparison of Oxygen Levels Stored in Different Bags

One unit of blood was collected from a healthy adult and immediately split into 3 equal parts. One part was stored in a 1000 mL capacity polyvinyl chloride (PVC) bag (Transfer Pack, Baxter Healthcare Corporation, Fenwal Division, Deerfield, Ill.), one part in a 1000 mL capacity PL732 bag (Lifecell, Tissue Culture Flask, Nexell Therapeutics Inc, Irvine, Calif.), and one part in a 1000 mL Teflon bag (American Flouriseal, Gaithersburg, Md.). All 3 bags had approximately the same surface area. After all free air was removed from each bag, all three were stored at room temperature and rocked gently in a platelet incubator/agitator (Helmer Labs Inc, Noblesville, Ind.). Oxygen tension and hemoglobin oxygen saturation were measured in each bag after 0, 1, 2, 3, 4, 6, and 8 hours of storage (i-STAT Portable Clinical Analyzer, i-STAT Corporation, East Windsor, N.J.).

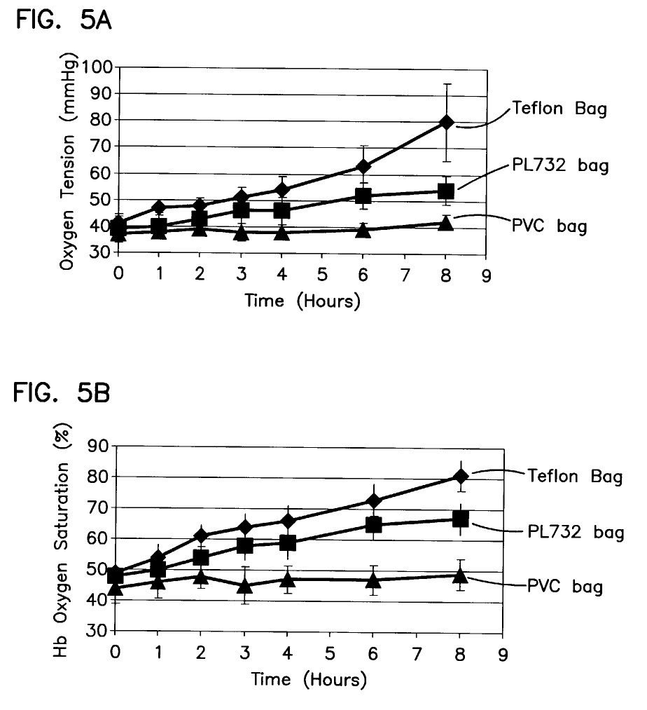

Oxygen tension and hemoglobin oxygen saturation levels are seen in FIGS. 5A and 5B. As seen there, both oxygen tension and hemoglobin oxygen saturation levels rose above baseline levels in all 3 bags over 8 hours of storage. The values shown in FIG. 5 are the mean±one standard error (n=6). Oxygen levels increased most rapidly in Teflon bags and least rapidly in PVC bags. Oxygen tension and hemoglobin oxygen saturation levels were greater in blood stored in PL732 bags than PVC bags at hours 3 through 8. Oxygen tension and hemoglobin oxygen saturation levels in blood stored in Teflon bags were greater than levels in blood stored in PVC bags at hours 1 through 8. Hemoglobin oxygen saturations were greater in blood stored in Teflon bags than in PL732 bags at hour 8 (p=0.03), but oxygen tensions were not (p=0.07).

In summary, incubation of blood from normal donors in PVC, PL732, and Teflon bags for a total of 8 hours revealed that the hemoglobin oxygen saturations increased from baseline levels of 44±12% to 48±10%, 54±13%, and 61±9%, respectively, after 2 hours and 49±12%, 67±13%, and 81±12% respectively after 8 hours.

The storage of blood in oxygen permeable bags may increase hemoglobin oxygen saturation to levels that are high enough to allow effective filtration of sickle cell trait donor RBC components. Many RBC storage bags are made from PVC, which is relatively impermeable to oxygen. However, bags made from Teflon or PL732 were more oxygen permeable and storage of blood for 8 hours in Teflon bags increased hemoglobin oxygen saturation to levels that likely would allow sickle trait donor blood to successfully filter.

### EXAMPLE #6

### Effect of Storage Bag Air on Blood Oxygen Levels

Since preliminary studies suggested that the presence of air in blood storage bags influenced the state of oxygenation, the effect, on oxygen levels, of adding different volumes of bulk air to blood storage bags was assessed. One unit of blood was collected from a healthy adult and was divided into 3 parts. Each part was placed into a 1000 mL capacity bag (PL732, Baxter). After all air was expressed from each bag, 0, 30, or 60 mL of air was added to each bag. The bags were stored at room temperature and rocked gently in a platelet incubator/agitator (Helmer). Oxygen tension and hemoglobin oxygen saturation in each bag were measured after 0, 1, 2, 3, 4, 6, and 8 hours of storage (i-STAT).

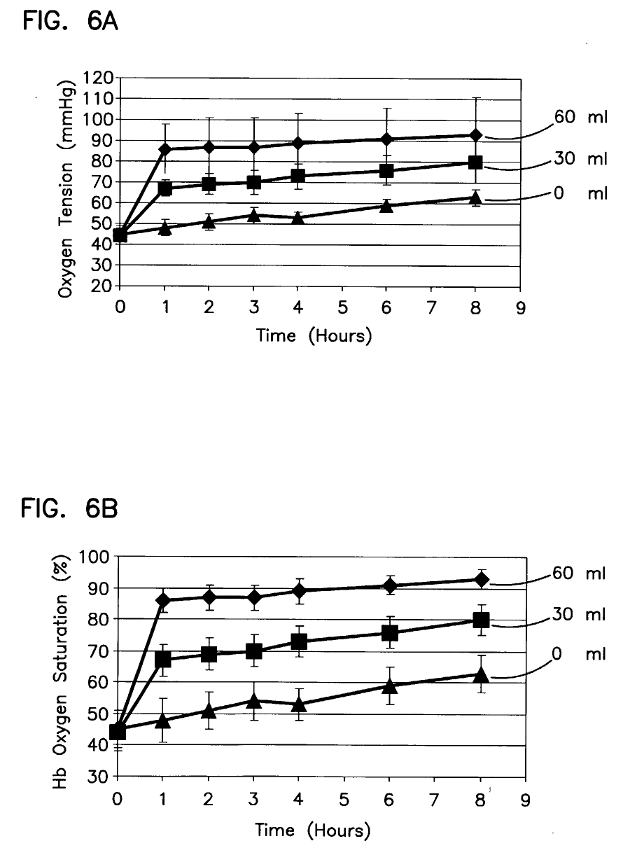

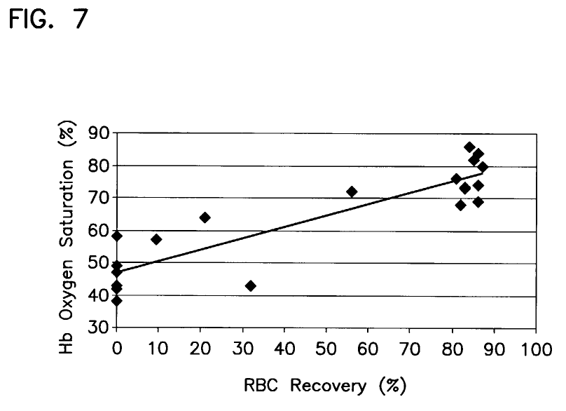

Oxygen tensions (FIG. 6A) and hemoglobin oxygen saturations (FIG. 6B) were compared among blood components stored with 0, 30, or 60 mL of added air. The values shown in FIGS. 6A and 6B are the mean±one standard error (n=6). Blood stored with 30 mL or 60 mL of added air had oxygen tensions and hemoglobin oxygen saturations after 1 hour of storage that were much greater than baseline levels. These levels then rose slowly from hours 1 through 8. In contrast, blood stored without added air had oxygen tensions and oxygen saturation levels that rose slowly over hours 1 through 8. After one hour of storage the oxygen tension and hemoglobin oxygen saturation was greater in components stored with 30 mL and 60 mL of added air than in components stored without air (p<0.0001). Oxygen tension and hemoglobin oxygen saturation levels in blood with added air remained greater than blood stored without air through hour 8. Oxygen tensions and hemoglobin oxygen saturations were greater in components stored with 60 mL of added air than in components with 30 mL of air at hours 1 through 8 (p<0.01).

### EXAMPLE #7

### Filtration of Sickle Cell Trait Donor Blood

One unit of blood from sickle cell trait donors was collected into a bag set that included CP2D anticoagulant, AS-3 additive solution, and an RBC leukocyte reduction filter (RCM1, Leukotrap RC System, Medsep Corporation, Pall Medical, Covina, Calif.). The bags were rocked during collection (Sebra, Tucson, Ariz.). The collected blood from each donor was divided into two parts. One part was placed in a 1000 mL PL732 bag (Nexell) with 60 mL of air added and the second part was transferred to a 600 mL PVC bag (Baxter). Both parts were stored for 2 hours at room temperature in a platelet incubator/agitator (Helmer). Oxygen tension and hemoglobin oxygen saturation of blood in each bag were measured before and after the 2-hour incubation period (i-STAT). (Table 7).

TABLE 7
Age, race, gender, and sickle hemoglobin (hemoglobin S)
concentration in the donors with sickle cell trait

| Number | Age (years) | Race | Gender | Hemoglobin S (%) |
|---|---|---|---|---|
| 1 | 44 | Black | male | 33.7 |
| 2 | 47 | Black | female | 39.0 |
| 3 | 46 | Black | male | 38.8 |
| 4 | 23 | Black | female | 37.5 |
| 5 | 25 | Black | female | 39.1 |
| 6 | 19 | Black | male | 38.5 |
| 7 | 44 | Black | female | 36.8 |
| 8 | 48 | Black | female | 38.4 |
| 9 | 54 | Black | female | 33.3 |
| 10 | 51 | Black | male | 38.1 |

Packed RBCs were prepared by centrifugation of the whole blood and RBC components were filtered according to the manufacturer's instructions, with the exception that only one half of the AS-3 additive solution (50 mL) was added to each packed RBC component. RBC components were allowed to filter for up to 120 minutes. Components were defined as filtering completely if all RBCs drained from the upper filtration bag and to the final RBC storage bag. The time to complete filtration was recorded. Components that did not completely filter within 120 minutes were considered filter failures. After filtration was complete or after 120 minutes RBC recoveries were calculated. For units that filtered completely, residual white blood cell (WBC) counts were measured by flow cytometery (LuecoCount Reagent, Becton Dickinson Biosciences, Immunocytometry Systems, San Jose, Calif.). These results are seen in Table 8 below.

TABLE 8
Comparison of the filtration of sickle cell trait RBC components agitated for 2 hours
in PVC bags without added air and in PL732 bags with 60 mL of added air

| | RBCs Stored in PVC bags without air | | | | | RBCs Stored in PL732 Bags with 60 mL of added air | | | | |
|---|---|---|---|---|---|---|---|---|---|---|
| | | | | RBC | Residual | | | Filtration | RBC | Residual |
| | pO₂ | Hb O₂ Sat | Filtration | Recovery | WBC Counts | pO₂ | Hb O₂ Sat | Time | Recovery | WBC Counts |
| No. | (mmHg) | (%) | Time (Min) | (%) | (× 10⁶) | (mmHg) | (%) | (Min) | (%) | (× 10⁶) |
| 1 | 49 | 73 | 38 | 83 | 0.01 | 64 | 86 | 16 | 84 | 0.03 |
| 2 | 38 | 47 | >120 | 0 | NA | 54 | 69 | 29 | 86 | 0.06 |
| 3 | 38 | 49 | >120 | 0 | NA | 56 | 74 | 17 | 86 | 0.02 |
| 4 | 36 | 43 | >120 | 32 | NA | 56 | 73 | 14 | 83 | 0.06 |
| 5 | 43 | 57 | >120 | 10 | NA | 65 | 80 | 22 | 87 | 0.1 |
| 6 | 48 | 64 | >120 | 21 | NA | 67 | 82 | 25 | 85 | 0.2 |
| 7 | 37 | 43 | >120 | 0 | NA | 56 | 72 | >120 | 56 | NA |
| 8 | 36 | 42 | >120 | 0 | NA | 54 | 68 | 43 | 82 | 0.01 |
| 9 | 33 | 38 | >120 | 0 | NA | 59 | 76 | 20 | 81 | 0.03 |
| 10 | 44 | 58 | >120 | 0 | NA | 70 | 84 | 15 | 86 | 0.01 |
| Avg. | 40 ± 5 | 51 ± 11 | | 15 ± 26 | | 60 ± 6 | 76 ± 6 | | 82 ± 9 | |

Hb = hemoglobin
Min = minutes
pO₂ = oxygen tension
NA = not applicable

As seen in Table 8, the hemoglobin oxygen saturations in one half unit of blood stored in bags with 60 mL of added bulk air increased from baseline levels of 49%±10% (values not included in Table 8, but seen in FIG. 6) to 76%±6% (p<0.001), but in control components without added air, oxygen saturations remained stable after 2 hours of storage (51±11%, p=0.06) (Table 8). Nine of ten components stored with 60 mL of added air filtered completely in 22±9 minutes (range 14 to 43 minutes). For all 9 components stored with 60 mL of added air that filtered completely the post-filtration white blood cell counts were less than 1×10⁶ and the RBC recovery was greater than 81% (mean=84%±2%). In contrast, 9 of 10 control components incubated in PVC bags without added air did not filter completely. The filtration time of the control component that filtered completely was 38 minutes. The RBC recovery was greater for components stored in PL732 bags with added air than with components stored in PVC bags without air (82%±9% vs 15%±26%, p<0.001).

### EXAMPLE #8

### Confirmation of Effect of Hemoglobin Oxygen Saturation

To demonstrate that the filtration of sickle cell trait donor RBCs was improved due to increased hemoglobin oxygen saturation levels and not due to another factor related to by bag type, whole blood units from 3 sickle trait donors were divided into two parts. One part was placed in a PL732 bag with 60 mL of added air and one part was placed in a PVC bag with 60 mL of added air. All 3 components that were incubated in PVC bags with 60 mL air filtered completely as did components incubated in PL732 bags with 60 mL of air (Table 9).

TABLE 9
Comparison of the filtration of sickle cell trait RBC components that were agitated for 2 hours
with 60 mL of added air but in different types of storage bags

| RBCs Stored in PVC bags without air | | | | | | RBCs Stored in PL732 Bags with 60 mL of added air | | | | |
|---|---|---|---|---|---|---|---|---|---|---|
| | | | | RBC | Residual | | | Filtration | RBC | Residual |
| | pO₂ | Hb O₂ Sat | Filtration | Recovery | WBC Counts | pO₂ | Hb O₂ Sat | Time | Recovery | WBC Counts |
| No. | (mmHg) | (%) | Time (Min) | (%) | (× 10⁶) | (mmHg) | (%) | (Min) | (%) | (× 10⁶) |
| 1 | 56 | 70 | 29 | 93 | 0.01 | 61 | 76 | 18 | 87 | 0.01 |
| 2 | 88 | 93 | 13 | 80 | 0.13 | 98 | 94 | 14 | 80 | 0.01 |
| 3 | 72 | 85 | 15 | 76 | 0.01 | 69 | 84 | 15 | 83 | 0.01 |
| Avg. | 72 ± 16 | 83 ± 12 | 19 ± 9 | 83 ± 9 | | 77 ± 19 | 85 ± 9 | 16 ± 2 | 83 ± 4 | |

Hb = hemoglobin
Min = minutes
pO₂ = oxygen tension

The above specification, examples and data provide a complete description of the manufacture and use of the composition of the invention. Since many embodiments of the invention can be made without departing from the spirit and scope of the invention, the invention resides in the claims hereinafter appended.

We claim:

1. A method of reducing leukocytes in whole blood comprising:
(a) collecting whole blood from a donor;
(b) increasing the oxygen level of said whole blood, wherein said whole blood comprises RBC component and a remainder component;
(c) filtering said RBC component to reduce the amount of leukocytes.

2. The method of claim 1, wherein increasing the oxygen level of said blood comprises the use of an oxygen permeable bag for collection of said whole blood.

3. The method of claim 2, wherein said oxygen permeable bag comprises polytetrafluoroethylene, polyvinyl chloride, or multi-layer film of polystyrene and polyolefin.

4. The method of claim 2, wherein air is added to said oxygen permeable bag before said whole blood is collected therein.

5. The method of claim 4, wherein the amount of air added is between about 30 mL and about 150 mL.

6. The method of claim 5, wherein said amount of air is about 120 mL.

7. The method of claim 2, wherein increasing the oxygen level of said blood further comprises agitating the oxygen permeable bag holding the collected whole blood.

8. The method of claim 3, wherein the oxygen permeable bag has a capacity between about 2 and about 3 L.

9. The method of claim 2, wherein increasing the oxygen level of said blood further comprises storing the collected blood in said oxygen permeable bag for longer than about 3 days.

10. The method of claim 1, wherein said oxygen level is increased through use of a gas permeable bag, through use of gas permeable tubing, by adding oxygen gas or air directly to said collected whole blood, by agitation of the collected whole blood, by increasing the storage time of said collected whole blood, or by combinations thereof.

11. The method of claim 10, wherein said oxygen level is increased through use of gas permeable tubing.

12. The method of claim 11, wherein said gas permeable tubing comprises fluoroethylenepropylene.

13. The method of claim 11, wherein said gas permeable tubing has a serpentine pathway.

14. The method of claim 11, wherein said gas permeable tubing comprises at least two discrete portions.

15. The method of claim 14, wherein said two discrete portions comprise a blood line and a gas line.

16. The method of claim 1, further comprising adding preservative to said whole blood.

17. The method of claim 16, wherein said preservative is added to said whole blood prior to increasing said oxygen level.

18. The method of claim 17, wherein said preservative is added to said whole blood after increasing said oxygen level.

19. The method of claim 1, further comprising separating said whole blood into said RBC component and said remainder component.

20. The method of claim 19, further comprising adding preservative to said RBC component.

21. The method of claim 20, wherein said preservative is added to said RBC component prior to increasing said oxygen level.

22. The method of claim 20, wherein said preservative is added to said RBC component after increasing said oxygen level.

23. The method of claim 1, where said oxygen level of said whole blood is increased at least about 50 mm Hg.

24. The method of claim 23, wherein said oxygen level of said whole blood is increased to at least about 70 mm Hg.

25. The method of claim 24, wherein said oxygen level of said whole blood is increased to at least about 90 mm Hg.

26. A method of reducing leukocytes in whole blood comprising:
(a) collecting whole blood from a donor, wherein said whole blood comprises RBC component and a remainder component;
(b) separating said whole blood into said RBC component and said remainder component;
(c) increasing the oxygen level of said RBC component;
(d) filtering said RBC component to reduce the amount of leukocytes.

27. The method of claim 26, wherein said oxygen level is increased through use of a gas permeable bag, through use of gas permeable tubing, by adding oxygen gas or air directly to said collected whole blood, by agitation of the collected whole blood, by increasing the storage time of said collected whole blood, or by combinations thereof.

28. The method of claim 27, further comprising adding preservative to said RBC component.

29. The method of claim 28, wherein said preservative is added to said RBC component prior to said oxygen level being increased.

30. The method of claim 29, wherein said preservative is added to said RBC component after said oxygen level has been increased.

31. A gas permeable tubing for use in a method of reducing leukocytes comprising:
(a) a first sheet;
(b) a second sheet positioned adjacent to said first sheet
wherein said second sheet comprises a gas permeable material, and said first sheet is fused with said second sheet.

32. The gas permeable tubing of claim 31, wherein said second sheet comprises fluoroethylenepropylene.

33. The gas permeable tubing of claim 31, wherein the fusion of said first sheet with said second sheet forms a blood line.

34. The gas permeable tubing of claim 31 further comprising a third sheet positioned adjacent to said second sheet.

35. The gas permeable tubing of claim 34, wherein said third sheet is fused with said second sheet.

36. The gas permeable tubing of claim 35, wherein fusion of said second sheet with said third sheet forms a gas line.

37. The gas permeable tubing of claim 34, wherein said second sheet comprises fluoroethylenepropylene.

38. The gas permeable tubing of claim 34, wherein said first sheet, said second sheet, and said third sheet comprise fluoroethylenepropylene.
